(19) United States
(12) Patent Application Publication
(10) Pub. No.: US 2009/0099371 A1
(43) Pub. Date: Apr. 16, 2009

(54) PROCESS FOR THE PREPARATION OF AMORPHOUS ATORVASTATIN CALCIUM SALT

(76) Inventors: Runjhun Gupta, Uttar Pradesh (IN); Khushi Ram, Uttar Pradesh (IN); Paramvir Bhadwal, Uttar Pradesh (IN); Rajesh Kumar Thaper, Uttar Pradesh (IN); Sushil Kumar Dubeu, Uttar Pradesh (IN)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/162,409

(22) PCT Filed: Jan. 31, 2006
(86) PCT No.: PCT/IN06/00036

§ 371 Date: Dec. 1, 2008

(51) Int. Cl. C07D 207/34

(52) U.S. Cl. 548/537

(57) ABSTRACT

Disclosed is a novel process for preparing pure amorphous form of Atorvastatin employing a suitable solvent system selected from water, water-miscible solvents or water-immiscible solvents or a mixture thereof.

### FIELD OF THE INVENTION

This invention, in general relates to the field of HMG Coenzyme A reductase inhibitors, in particular to Atorvastatin. More specifically the present invention provides a novel and industrially feasible process to achieve a pure form of amorphous Atorvastatin calcium employing suitable solvent system.

### BACKGROUND OF THE INVENTION

[R(R*,R*)]-2-(4-Fluorophenyl)-β,δ-dihydroxy-5-(1-methylethyl)-3-phenyl-4-[phenyl amino)carbonyl]-1H-pyrrole-1-heptanoic acid, commonly known as Atorvastatin is known to be a therapeutically useful compound. Atorvastatin calcium, a synthetic HMG-CoA reductase inhibitor, is used for the treatment of hyperlipidemia and hypercholesterolemia, both of which are risk factors for arteriosclerosis and coronary heart disease. For use in the treatment of aforementioned diseases, open dihydroxy carboxylic acid, lactone and various salt forms of Atorvastatin have been synthesized.

U.S. Pat. No. 4,681,893 discloses certain trans-6-[2-(3- or 4-carboxamido substituted-pyrrol-1-yl)alkyl]-4-hydroxy-pyran-2-ones, which include trans(±)-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide, whereas U.S. Pat. No. 5,273,995 discloses that the R-enantiomer of the ring-opened acid form of trans-5-(4-fluorophenyl)-2-(1-methylethyl)-N,4-diphenyl-1-[2-(tetrahydro-4-hydroxy-6-oxo-2H-pyran-2-yl)ethyl]-1H-pyrrole-3-carboxamide has surprising inhibition of the biosynthesis of cholesterol. Atorvastatin in its calcium salt form, i.e. [R(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) having formula I; is more suited for

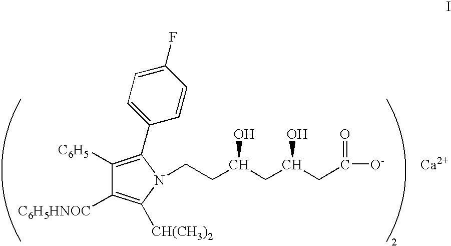

developing formulations and has been recommended as a drug.

U.S. Pat. Nos. 5,003,080; 5,097,045; 5,103,024; 5,124,482; 5,149,837; 5,155,251; 5,216,174; 5,245,047; 5,248,793; 5,280,126; 5,342,952 and 5,397,792 disclose various processes and key intermediates for preparing Atorvastatin.

One of the disadvantages of processes described in the above-mentioned patents is the preparation of (R-cis)-1,1-dimethylethyl-6-[2[2-(4-fluorophenyl)-5-(1-methylethyl)-3-phenyl-4-phenylcarbamoyl-1H-pyrrol-1-yl]ethyl]-2,2-dimethyl-1,3-dioxane-4-acetate, wherein a binary or ternary solvent system is used in the condensation step. This makes the recovery of the solvent very difficult at the plant scale and thus these processes are not commercially viable on an industrial scale.

Another disadvantage of the processes discussed in these patents is the inconsistency in the polymorphic nature of the final product i.e. Atorvastatin calcium. The final product is in the form of mixture of crystalline and amorphous forms, which has unsuitable filtration and drying characteristics and is not suitable for large-scale production.

It is known that the amorphous forms of a number of pharmaceutical substances exhibit different dissolution characteristics and bioavailability patterns compared to crystalline forms (Konno T., Chem. Phar. Bull. 1990, 38, 2003-2007). For some therapeutic indications the bioavailability is one of the key parameters determining the form of the substance to be used in a pharmaceutical formulation. There is a constant need for processes which enable the preparation of Atorvastatin in an amorphous form without simultaneous formation of crystalline forms, or which will enable the conversion of the crystalline forms into the amorphous form.

Atorvastatin calcium is very slightly water-soluble, and it has been found that in comparison to an amorphous form, crystalline forms are less readily soluble and adversely affect the bioavailability of Atorvastatin in the body.

PCT application WO 97/03959 discloses novel crystalline forms of Atorvastatin calcium designated as form I, form II and form IV and processes for their preparation. PCT application WO 97/03960 and U.S. Pat. No. 6,274,740 describe the processes for the preparation of amorphous form, by conversion of the crystalline form of Atorvastatin. The process disclosed therein comprises dissolving Atorvastatin crystalline form I in a non-hydroxylic solvent like tetrahydrofuran or mixtures of tetrahydrofuran and toluene. This process involves complete removal of the solvent under high temperature (about 90° C.) and high vacuum (about 5 mm). The exposure of the material to high temperature for several days leads to degradation of the product. This makes the process very inconvenient to operate on a large scale. Slow removal of solvent on a manufacturing scale renders this process less productive.

PCT application WO 00/71116 describes the process for the preparation of amorphous Atorvastatin, which involves dissolving crystalline form in non-hydroxylated solvent followed by precipitation of amorphous Atorvastatin by adding non-polar hydrocarbon solvent. In this case high levels of hydrocarbon are necessary to obtain the desired product. A similar approach is described in PCT application WO 01/42209, which describes conversion of the crystalline form of Atorvastatin to the amorphous form by dissolving in a variety of solvents including both non-hydroxylated solvents and lower alcohols, followed by precipitation with solvents in which Atorvastatin is insoluble, like non-polar hydrocarbons or aliphatic ethers. This process also is not recommended for commercial production of amorphous Atorvastatin calcium due to the use of a large excess of diethyl ether, which is not safe on a commercial scale. PCT application WO 2005/092852 describes the preparation of amorphous Atorvastatin starting from a diol-protected ester derivative of Atorvastatin, which is converted first to crude Atorvastatin calcium, purified to crystalline Atorvastatin calcium and then treated with tetrahydrofuran and cyclohexane to get the desired product.

Whereas, PCT application WO 01/28999 describes the purification of crude amorphous Atorvastatin calcium by dissolving crude amorphous material in a large excess of boiling ethanol or 2-propanol and filtering the hot solution and recovering the material at low temperature. The hot solution is difficult to filter on the industrial scale.

The present invention provides a novel and industrially viable process for preparing Atorvastatin in pure amorphous form to avoid the drawback associated with the prior art.

### SUMMARY OF THE INVENTION

It is a principal aspect of the present invention to provide a novel process for the preparation of pure amorphous form of Atorvastatin calcium employing a suitable solvent system.

The aspects of the present invention are further described hereinafter in different preferred embodiments in accordance with the best mode of the invention, however the invention is not restricted to the described embodiments.

In accordance with one preferred embodiment of the present invention, there is provided a novel process for preparation of pure amorphous form of Atorvastatin calcium, wherein the process comprises the steps of:
(a) treating a solution of 7-[2-(4-fluorophenyl)-5-isopropyl-3-phenyl-4-phenylcarbamoyl-pyrrol-1-yl]-3,5-dihydroxy heptanoic acid t-butyl ester of formula (II) in water or water-miscible solvent with a base;
(b) adding aqueous solution of calcium salt to the solution of step (a) to prepare Atorvastatin calcium;
(c) adding organic solvent;
(d) separating the organic layer and evaporating to afford crude amorphous Atorvastatin calcium as a foamy solid;
(e) adding anti-solvents to the solid of step (d) in which Atorvastatin calcium is insoluble; and
(f) separating the resultant precipitate to obtain pure amorphous Atorvastatin calcium.

In accordance with a further embodiment of present invention, there is provided a novel process for the preparation of pure amorphous form of Atorvastatin calcium, wherein the foamy solid obtained according to step (d) is optionally stirred by using tetrahydrofurane followed by adding anti solvent to obtain the amorphous form of Atorvastatin calcium.

In accordance with another embodiment of the present invention, the process comprises treating a solution of 7-[2-(4-fluorophenyl)-5-isopropyl-3-phenyl-4-phenylcarbamoyl-pyrrol-1-yl]-3,5-dihydroxy heptanoic acid t-butyl ester of formula (II) in a water-miscible solvent with a base, wherein said water-miscible solvent is selected from methanol, isopropyl alcohol and ethanol.

In accordance with another embodiment of the present invention, the process comprises treating a solution of 7-[2-(4-fluorophenyl)-5-isopropyl-3-phenyl-4-phenylcarbamoyl-pyrrol-1-yl]-3,5-dihydroxy heptanoic acid t-butyl ester of formula (II) in a water-immiscible solvent with aqueous base, wherein the water-immiscible solvent is selected from methyl ethyl ketone or methyl isobutyl ketone.

In accordance with one other embodiment of the present invention, there is provided a process for preparing amorphous form of Atorvastatin, wherein the calcium salt used to prepare a salt of Atorvastatin is selected from calcium chloride, calcium hydroxide, calcium acetate or calcium 2-ethyl hexanoate, and preferably calcium 2-ethyl hexanoate.

In accordance with yet another embodiment of the present invention, there is provided a process to prepare pure form of amorphous Atorvastatin, wherein the compound 7-[2-(4-fluorophenyl)-5-isopropyl-3-phenyl-4-phenylcarbamoyl-pyrrol-1-yl]-3,5-dihydroxy heptanoic acid t-butyl ester of formula (II) is prepared by reacting 1,1-dimethylethyl 6-(2-aminoethyl)-2-phenyl-1,3-dioxane-4-acetate (IV) with 4-fluoro-α-[2-methyl-1-oxopropyl]-γ-oxo-N,β-diphenylbenzenebutaneamide (V) in the presence of pivalic acid and solvent selected from cyclic or acyclic hydrocarbon to afford t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III), treating the compound of formula III with mineral acid to get the compound of formula II, which is further used in the process for preparing amorphous form of Atorvastatin as described in the above-mentioned embodiments.

In accordance with still another embodiment of the present invention, there is provided a process to prepare pure form of amorphous Atorvastatin, wherein the compound 7-[2-(4-fluorophenyl)-5-isopropyl-3-phenyl-4-phenylcarbamoyl-pyrrol-1-yl]-3,5-dihydroxy heptanoic acid t-butyl ester of formula (II) is prepared by reacting 1,1-dimethylethyl 6-(2-aminoethyl)-2-phenyl-1,3-dioxane-4-acetate (IV) with 4-fluoro-α-[2-methyl-1-oxopropyl]-γ-oxo-N,β-diphenylbenzene butaneamide (V) in the presence of pivalic acid and cyclohexane to afford t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III), treating t-butyl (6 {2-[2-(4-fluorophenyl)-5-isopropyl-3-phenyl-4-phenylcarbamoylpyrrol-1-yl]ethyl}-2,2-dimethyl[1,3]dioxan-4-yl)acetate (III) with mineral acid to get the compound of formula II, which is further used in the process for preparing amorphous form of Atorvastatin as described in the above-mentioned embodiments.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and examples.

The disclosed embodiments of the present invention deal with a process for the preparation of pure amorphous Atorvastatin calcium employing suitable solvent systems.

The process for the preparation of pure amorphous Atorvastatin using a compound of formula II is disclosed in the present invention, wherein the process comprises contacting a compound of formula II with water, solvent or mixture thereof at room temperature, adding an aqueous solution of base, wherein base is selected from the group including, but not limited to, alkali or alkaline earth metal hydroxide, preferably sodium hydroxide, potassium hydroxide, lithium hydroxide or calcium hydroxide, and more preferably sodium hydroxide. The solvent used in the hydrolysis step is selected from the group including, but not limited to, a water-miscible solvent such as an alcoholic solvent like methanol, isopropyl alcohol and ethanol, and a water-immiscible solvent such as methyl ethyl ketone and methyl isobutyl ketone. After the complete hydrolysis, the reaction mixture is cooled and then an aqueous solution of calcium salt is added, wherein the calcium salt is selected from the group including, but not limited to, calcium acetate, calcium chloride, calcium hydroxide or calcium 2-ethyl hexanoate, and preferably calcium acetate and calcium 2-ethylhexanoate. The addition of the source of calcium ions is carried out at a temperature from 40-60° C. Atorvastatin calcium thus prepared is then extracted with the organic solvent, wherein the organic solvent is selected from the group including, but not limited to, ethyl acetate, xylene, toluene and methyl isobutyl ketone (MIBK), and preferably ethyl acetate. This extracted organic layer is washed twice with water and then evaporated to dryness to get a foamy solid, which is the crude amorphous form of Atorvastatin calcium as characterized by powder X-ray diffraction. The powder XRD of foamy solid indicates that the product is 100% amorphous. This foamy solid is ten taken into anti-solvent in which the product is less soluble or insoluble at room temperature, or is optionally first dissolved in tetrahydrofuran at room temperature and then added to anti-solvents or vice versa. The anti-solvents are selected from the group including, but not limited to, methyl t-butyl ether (MTBE), cyclohexane, hexane, heptane, octane, isopropyl alcohol, diisopropyl ether and diethyl ether or mixture thereof, and preferably cyclohexane and methyl isobutyl ketone.

A compound of formula II is prepared by the process which comprises the reaction of a compound of formula IV with a compound of formula V in presence of pivalic acid and solvent to get an intermediate III, which is further treated with a mineral acid, wherein the mineral acid is selected from the group including, but not limited to, hydrochloric acid and sulfuric acid, to afford a compound of formula II.

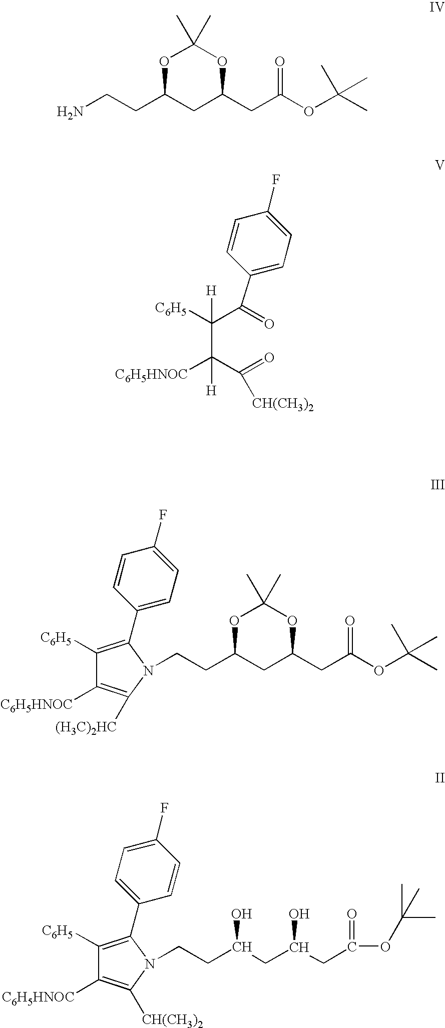

The solvent used herein is selected from the group including, but not limited to, a cyclic hydrocarbon such as cyclohexane. and an acyclic hydrocarbon such as hexane, pentane, and heptane. In the preparation of an intermediate III, a single solvent is used, whereas in the prior art, a binary or ternary system is used. The advantages of using a single solvent system are the recovery and reuse of the solvent in the process, and the ease of separating the final product III relative to the binary or ternary systems. In the latter case the workup is much more tedious, time consuming, and environmentally and economically unfeasible at the industrial scale, which problems are eliminated by the instant process.

Having thus described the various methods for the preparation of the amorphous form of Atorvastatin calcium according to the present invention, the following examples are provided to illustrate specific embodiments of the present invention. They are, however, not intended to limit the scope of the present invention in any way.

### EXAMPLE 1

Dihydroxy ester (II) (5 g) was taken in water (50 ml) and sodium hydroxide (0.35 g) was added. The temperature was raised to 75-80° C. and the reaction mass was stirred and then cooled. Calcium acetate (1.00 g) was added and stirred for 1 hr and pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether or isopropyl alcohol (50 ml) and stirred. Contents were filtered and dried to yield amorphous Atorvastatin calcium.

### EXAMPLE 2

Dihydroxy ester (II) (5 g) was taken in water (50 ml) and sodium hydroxide (0.35 g) was added. The temperature was raised to 75-80° C. and the reaction mass was stirred and then cooled. Calcium acetate solution (1.00 g) was added and stirred for 1 hr and pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in a mixture of cyclohexane and t-butyl methyl ether (50 ml) and stirred. Contents were filtered and dried to yield amorphous Atorvastatin calcium.

### EXAMPLE 3

Dihydroxy ester (II) (5 g) was taken in water (50 ml) and sodium hydroxide (0.35 g) was added. The temperature was raised to 75-80° C. and the reaction mass was stirred for 12 hrs and then cooled. Calcium 2-ethyl hexanoate solution (2.20 g) was added and stirred for 1 hr. The pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether (50 ml) and stirred. Contents were filtered and dried to yield amorphous Atorvastatin calcium.

### EXAMPLE 4

Dihydroxy ester (II) (5 g) was taken in water (25 ml) and methyl ethyl ketone (25 ml). Sodium hydroxide (0.35 g) was then added. The temperature was raised to 60° C., and the reaction mass was stirred for 2 hrs. After completion of the reaction, calcium acetate (1.00 g in 5 ml water) was added and stirred, followed by the cooling of the reaction mixture to room temperature. Methyl ethyl ketone (25 ml) was added to extract the product from the aqueous layer. The organic layer was washed with water and then dried over sodium sulfate. Solvent was removed under vacuum to give a foamy solid residue. The foamy solid residue was taken in diisopropyl ether (50 ml) and stirred. Contents were filtered and dried to yield amorphous Atorvastatin.

### EXAMPLE 5

Dihydroxy ester (II) (5 g) was taken in water (25 ml) and methyl ethyl ketone (25 ml) followed by the addition of sodium hydroxide (0.35 g). The temperature of the reaction mixture was raised to 60° C., and the mixture was stirred for 2 hrs. After completion of the reaction, calcium acetate (1.00 g in 5 ml water) was added and stirred for 2 hrs. The reaction mixture was cooled to room temperature. The pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether or isopropyl alcohol or a mixture of cyclohexane and t-butyl methyl ether (50 ml) and stirred. Contents were filtered and dried to yield amorphous Atorvastatin.

### EXAMPLE 6

Dihydroxy ester (II) (5 g) was taken in water (25 ml) and methyl ethyl ketone (25 ml) followed by the addition of sodium hydroxide (0.35 g). The temperature of the reaction mixture was raised to 60° C., and stirred for 2 hrs. After completion of the reaction, calcium 2-ethyl hexanoate (2.20 g in 5 ml water) was added and stirred for 2 hrs. The reaction mixture was cooled to room temperature. The pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether, isopropyl alcohol or a mixture of cyclohexane and t-butyl methyl ether (50 ml) and stirred. Contents were filtered and dried to yield amorphous Atorvastatin.

### EXAMPLE 7

Dihydroxy ester (II) (5 g) was taken in water (25 ml) and methyl ethyl ketone (25 ml) followed by the addition of sodium hydroxide (0.35 g). The temperature of the reaction mixture was raised to 60° C., and the mixture was stirred for 2 hrs. After completion of the reaction, calcium acetate (1.00 g in 5 ml water) was added and stirred for 2 hrs. The reaction mixture was cooled to room temperature. The pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether, isopropyl alcohol or a mixture of cyclohexane and t-butyl methyl ether (50 ml) and stirred. Contents were filtered and dried to yield amorphous Atorvastatin.

### EXAMPLE 8

Dihydroxy ester (II) (5 g) was taken in methyl ethyl ketone (40 ml) followed by the addition of sodium hydroxide (0.35 g). The temperature of the reaction mixture was raised to 60° C., and the mixture was stirred for 2 hrs. After completion of the reaction, calcium 2-ethyl hexanoate (2.20 g in 5 ml water) was added and stirred for 2 hrs. The reaction mixture was cooled to room temperature. The pH was adjusted to 8. Ethyl acetate (40 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether, isopropyl alcohol or a mixture of cyclohexane and t-butyl methyl ether (50 ml) and stirred. Contents were filtered and dried to yield amorphous Atorvastatin.

### EXAMPLE 9

Dihydroxy ester (II) (5 g) was taken in water (25 ml) and methyl ethyl ketone (25 ml) followed by the addition of sodium hydroxide (0.35 g). The temperature of the reaction mixture was raised to 60° C., and the mixture was stirred for 2 hrs. After completion of the reaction, calcium acetate solution (1.00 g in 5 ml water) was added and stirred for 2 hrs. The reaction mixture was cooled to room temperature. The pH was adjusted to 8. Xylene (50 ml) was added to extract the product from the aqueous layer. The organic layer was washed with water and then dried over sodium sulfate. Solvent was removed under vacuum to give a foamy solid residue. The foamy solid residue was taken in diisopropyl ether, cyclohexane, t-butyl methyl ether, isopropyl alcohol or a mixture of cyclohexane and t-butyl methyl ether (50 ml) and stirred. Contents were filtered and dried to yield amorphous Atorvastatin.

### EXAMPLE 10

Dihydroxy ester (II) (5 g) was taken in water (50 ml) and methanol (5 ml). Sodium hydroxide (0.36 g, 1.1 mole eq.) was then added. The temperature was raised to 80° C. and the reaction mass was stirred for 3 hrs at this temperature. After completion of the reaction, the reaction mixture was cooled. Calcium acetate solution (1.02 g in 5 ml water) was added at this temperature, and the mixture was then stirred for 1 hr. Ethyl acetate (40 ml) was added to extract the product from the aqueous layer. The organic layer was washed with water and then dried over sodium sulfate. Solvent was removed under vacuum to give a foamy solid residue. To the residue, tetrahydrofuran (15 ml) was added. The residue was then stirred and stripped of the tetrahydrofuran under vacuum at 40-50° C. to get the solid foam. Tetarhydrofuran (15 ml) was added and stirred for dissolution, followed by the addition of this solution to a cyclohexane:t-butyl methyl ether mixture (75 ml: 75 ml) at room temperature. After the addition, the precipitated material was stirred for one hour. Contents were filtered and dried to yield amorphous Atorvastatin.

### EXAMPLE 11

### Preparation of Compound II

Diketo compound (V) (1.50 g) was taken in cyclohexane (10 ml) at room temperature. Amino ketal compound (IV) (1.00 g) in cyclohexane was added followed by the addition of pivalic acid (0.11 g). The reaction mixture was refluxed and after the completion of the reaction, the reaction mixture was cooled and the precipitate filtered. The material was dried in a vacuum oven at 50-55° C. for 2 hr (moisture content is approx. 0.5%) to get crude compound III, which was recrystallized in isopropyl alcohol to give pure compound III as a solid. Compound III (1.40 gm) was then taken into methanol (20 ml) and stirred, followed by the addition of dilute hydrochloric acid. The reaction mixture was warmed and stirred. After completion of the reaction, the reaction mixture was cooled and water was added. The precipitated material was filtered and dried under vacuum at 50-55° C. for 12 hrs to afford the desired product II.

While this invention has been described in detail with reference to certain preferred embodiments, it should be appreciated that the present invention is not limited to those embodiments. Rather, in view of the present disclosure, which describes the current best mode for practicing the invention, many modifications and variations would present themselves to those skilled in the art without departing from the scope and spirit of this invention.

1-18. (canceled)

19. A process for preparing pure amorphous form of Atorvastatin calcium comprising:
(a) treating with a base a solution of 7-[2-(4-fluorophenyl)-5-isopropyl-3-phenyl-4-phenylcarbamoyl-pyrrol-1-yl]-3,5-dihydroxy heptanoic acid t-butyl ester of formula (II) in water or a water-miscible solvent;
(b) adding an aqueous solution of calcium salt to the solution of step (a) to prepare Atorvastatin calcium;
(c) adding an organic solvent to the solution of step (a) to form an organic layer;
(d) separating the organic layer and evaporating to afford crude amorphous Atorvastatin calcium as a foamy solid;
(e) adding an anti-solvent in which Atorvastatin calcium is insoluble; and
(f) separating the resultant precipitate to obtain pure amorphous Atorvastatin calcium.

20. The process according to claim 19, wherein the foamy solid obtained according to step (d) is stirred with tetrahydrofurane followed by adding anti-solvent to obtain the amorphous form of Atorvastatin calcium.

21. The process according to claim 19, wherein the water-miscible solvent is selected from the group consisting of methanol, isopropyl alcohol and ethanol.

22. The process according to claim 19, wherein the calcium salt is selected from the group consisting of calcium chloride, calcium hydroxide, calcium acetate and calcium 2-ethyl hexanoate.

23. The process according to claim 22, wherein the calcium salt is calcium 2-ethyl hexanoate.

24. The process according to claim 19, wherein the organic solvent is selected from the group consisting of ethyl acetate, xylene, toluene and methyl isobutyl ketone.

25. The process according to claim 19, wherein the anti-solvent is selected from the group consisting of methyl t-butyl ether (MTBE), cyclohexane, hexane, heptane, octane, isopropyl alcohol, diisopropyl ether, diethyl ether, and a mixture thereof.

26. A process for preparing pure amorphous form of Atorvastatin calcium, which comprises:
(a) reacting an aqueous base with a solution of 7-[2-(4-fluorophenyl)-5-isopropyl-3-phenyl-4-phenylcarbamoyl-pyrrol-1-yl]-3,5-dihydroxy heptanoic acid t-butyl ester of formula (II) in a water-immiscible solvent;
(b) adding an aqueous solution of calcium salt to the solution of step (a) to prepare Atorvastatin calcium;
(c) adding an organic solvent to the solution of step (a) to form an organic layer;
(d) separating the organic layer and evaporating to afford crude amorphous Atorvastatin calcium as a foamy solid;
(e) adding an anti-solvent in which Atorvastatin calcium is insoluble; and
(f) separating the resultant precipitate to obtain pure amorphous Atorvastatin calcium.

27. The process according to claim 26, wherein the water-immiscible solvent is selected from the group consisting of methyl ethyl ketone and methyl isobutyl ketone.

28. The process according to claim 26, wherein the calcium salt is selected from the group consisting of calcium chloride, calcium hydroxide, calcium acetate and calcium 2-ethyl hexanoate.

29. The process according to claim 28, wherein the calcium salt is calcium 2-ethyl hexanoate.

30. The process according to claim 26, wherein the organic solvent is selected from the group consisting of ethyl acetate, xylene, toluene and methyl isobutyl ketone.

31. The process according to claim 26, wherein the anti-solvent is selected from the group consisting of methyl t-butyl ether (MTBE), cyclohexane, hexane, heptane, octane, isopropyl alcohol, diisopropyl ether, diethyl ether and a mixture thereof.

32. The process of claim 19, wherein the compound 7-[2-(4-fluorophenyl)-5-isopropyl-3-phenyl-4-phenylcarbamoyl-pyrrol-1-yl]-3,5-dihydroxy heptanoic acid t-butyl ester is prepared by reacting 1,1-dimethylethyl 6-(2-aminoethyl)-2-phenyl-1,3-dioxane-4-acetate (IV) with 4-fluoro-α-[2-methyl-1-oxopropyl]-γ-oxo-N,β-diphenylbenzenebutaneamide (V) in the presence of pivalic acid and solvent to afford the t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III), and treating the t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III) with mineral acid.

33. The process according to claim 32, wherein the solvent is cyclic hydrocarbon or acyclic hydrocarbon.

34. The process according to claim 33, wherein the cyclic hydrocarbon is cyclohexane.

35. The process according to claim 33, wherein the acyclic hydrocarbon is selected from the group consisting of pentane, hexane, heptane and octane.

36. The process according to claim 32, wherein the mineral acid is hydrochloric acid or sulfuric acid.

37. The process of claim 26, wherein the compound 7-[2-(4-fluorophenyl)-5-isopropyl-3-phenyl-4-phenylcarbamoyl-pyrrol-1-yl]-3,5-dihydroxy heptanoic acid t-butyl ester is prepared by reacting 1,1-dimethylethyl 6-(2-aminoethyl)-2-phenyl-1,3-dioxane-4-acetate (IV) with 4-fluoro-α-[2-methyl-1-oxopropyl]-γ-oxo-N,β-diphenylbenzenebutaneamide (V) in the presence of pivalic acid and solvent to afford the t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III), and treating the t-butyl (6-{2-[2-(4-fluorophenyl)-5-isopropyl-3-phenyl-4-phenylcarbamoylpyrrol-1-yl]ethyl}-2,2-dimethyl[1,3]dioxan-4-yl)acetate (III) with mineral acid.

38. The process according to claim 37, wherein the solvent is cyclic hydrocarbon or acyclic hydrocarbon.

39. The process according to claim 38, wherein the cyclic hydrocarbon is cyclohexane.

40. The process according to claim 38, wherein the acyclic hydrocarbon is selected from the group consisting of pentane, hexane, heptane and octane.

41. The process according to claim 37, wherein the mineral acid is hydrochloric acid or sulfuric acid.
